(19) United States
(12) Patent Application Publication
(10) Pub. No.: US 2022/0233260 A1
(43) Pub. Date: Jul. 28, 2022

(54) SURGICAL TOOL

(71) Applicant: RIVERFIELD INC., Tokyo (JP)

(72) Inventors: Koki Shindo, Tokyo (JP); Kotaro Tadano, Tokyo (JP)

(73) Assignee: RIVERFIELD INC., Tokyo (JP)

(21) Appl. No.: 17/721,861

(22) Filed: Apr. 15, 2022

Related U.S. Application Data
(63) Continuation of application No. PCT/JP2020/035625, filed on Sep. 18, 2020.

(30) Foreign Application Priority Data
Oct. 17, 2019 (JP) 2019-190340

(51) Int. Cl. A61B 34/30; A61B 34/00

(52) U.S. Cl. CPC A61B 34/30; A61B 34/71

(57) ABSTRACT

A surgical tool includes a shaft, a treatment portion provided at a distal end of the shaft, and a body provided at a proximal end of the shaft. The body includes a driven portion that moves in direction parallel to the shaft, based on a first driving force from a surgical robot. A wire or rod that is connected through the shaft to the treatment portion. A lever that converts the first driving force to a second driving force, the second driving force being different from the first driving force, and a connection portion that is connected to the lever and to the wire or rod, and that transmits the second driving force to the wire or rod.

### CROSS-REFERENCE TO RELATED APPLICATION

This Application is a Continuation Application of International Application No. PCT/JP2020/035625 filed on Sep. 18, 2020, which is based on Japanese patent application No. 2019-190340 filed on Oct. 17, 2019 with the Japan Patent Office, the entire contents of each of which are incorporated by reference herein in their entireties.

### BACKGROUND

The present disclosure relates to a surgical tool used for medical robots.

In recent years, medical treatments using robots have been proposed for the purpose of reducing burdens on operators and reducing manpower at medical facilities. In such cases, a surgical tool is attached to the medical robot and a force is transmitted from the medical robot to the surgical tool to provide the medical treatment. However, when, for example, a size of the surgical tool changes, it becomes difficult to stably and smoothly operate the surgical tool, and difficult to prevent a deterioration in accuracy in estimating the force that will be applied by the surgical tool during treatment.

### SUMMARY

It is an aspect to provide a surgical tool with which it is possible to prevent deterioration in operability and to increase accuracy in estimating a force to be applied by the surgical tool, when a size of the surgical tool is changed.

According to an aspect of one or more embodiments, there is provided a surgical tool comprising a driven portion configured to move in a linear motion direction, based on a driving force; a power transmission portion configured to transmit the driving force for moving in the linear motion direction to a treatment portion configured to perform a medical treatment; a conversion portion configured to convert a first amount of movement of the driven portion in the linear motion direction to a second amount of movement different from the first amount of movement and to transmit the second amount of movement to the power transmission portion; and a body that includes the driven portion and the conversion portion, and that supports the treatment portion.

According to another aspect of one or more embodiments, there is provided a surgical tool comprising a shaft; a treatment portion provided at a distal end of the shaft; and a body provided at a proximal end of the shaft, wherein the body comprises a driven portion that moves in direction parallel to the shaft, based on a first driving force from a surgical robot; a wire or rod that is connected through the shaft to the treatment portion; a lever that converts the first driving force to a second driving force, the second driving force being different from the first driving force; and a connection portion that is connected to the lever and to the wire or rod, and that transmits the second driving force to the wire or rod.

### BRIEF DESCRIPTION OF THE DRAWINGS

The above and/or other aspects will become apparent from the following description of various embodiments and with reference to the following drawings, in which:

FIG. 1 is a diagram illustrating an overall configuration of a surgical tool according to various embodiments;

FIG. 2 is a diagram illustrating an example configuration of an attachment/detachment surface of a body of the surgical tool in FIG. 1, according to various embodiments;

FIG. 3 is a diagram illustrating example configurations of a driven portion, a connection portion, a conversion portion, and a power transmission portion of the surgical tool of FIG. 1, according to various embodiments;

FIG. 4 is a perspective view illustrating example configurations of the driven portion, the connection portion, the conversion portion, and the power transmission portion in FIG. 3, according to various embodiments;

FIG. 5 is a diagram illustrating an example configuration of the connection portion in FIGS. 3-4, according to various embodiments.

FIG. 6 is a diagram illustrating transmission of a driving force, according to various embodiments;

FIG. 7 is another diagram illustrating transmission of a driving force, according to various embodiments;

FIG. 8 is a diagram illustrating configurations of a driven portion, a connection portion, a conversion portion, and a power transmission portion of a surgical tool, according to various embodiments; and

FIG. 9 is a diagram illustrating the configuration of the connection portion in FIG. 8, according to various embodiments.

### DETAILED DESCRIPTION

In recent years, medical treatments using robots have been proposed for the purpose of reducing burdens on operators and reducing manpower at medical facilities. In the field of surgery, medical robots having a remotely operable arm with multi-degrees of freedom have been used to treat patients.

Such related art medical robots may have a configuration that allows attachment/detachment to/from the medical robot of a surgical tool that is used for treatment. A driving force in a linear motion direction may be transmitted from the medical robot to the surgical tool through a wire and a rod, arranged within the surgical tool, to allow treatment with a gripper, arranged at an end of the surgical tool.

It is advantageous for patients to have treatments with low invasiveness, in which a degree of invasiveness to patients is reduced, to provide an improved cosmetic outcome. Therefore, there is a desire for miniaturization and narrowing in diameter of the surgical tools.

At the same time, it is desirable that the medical robots be easy to use such that operators of such medical robots may spend less time to learn how to use the medical robot, to learn how to perform various operation procedures, and to be able to stably and smoothly handle the surgical tool.

For example, it has been desired to improve the accuracy in estimating the magnitude, the direction, and so on of an external force acting on the surgical tool based on information, such as the position and the driving force of an actuator that drives the surgical tool, and transmit the estimated external force to an operator remotely handling the surgical tool. The accuracy in estimating an external force depends on the signal to noise (S/N) ratio when the external force is detected and the resolution when an operation amount of the treatment portion (e.g., the gripper, a scissors, monopolar hooks, spatulas, etc.) is measured.

When the size of the treatment portion, for example, a gripper, a scissors, monopolar hooks, spatulas, etc., of the surgical tool is changed, the amount of operation of the treatment portion varies even though strokes generated by a driving force transmitted from the medical robot to the surgical tool have the same lengths. In addition, the magnitude of a force generated in the treatment portion also varies even though the magnitude of the driving force is the same.

Hence, when the size of the surgical tool attached to the medical robot, that is, for example, the size of the gripper, is changed, the S/N ratio and resolution also vary, affecting the accuracy in estimating the external force. In other words, there is a disadvantage in that it becomes difficult to stably and smoothly operate the surgical tool, and difficult to prevent a deterioration in accuracy in estimating the external force.

It is an aspect of one or more embodiments to provide a surgical tool with which it is possible to prevent deterioration in operability and it is possible to improve accuracy in estimating an external force due to a change in size of the surgical tool.

The surgical tool according to various embodiments may include a driven portion configured to move upon receipt of an external driving force for moving in a linear motion direction, a power transmission portion configured to transmit the driving force for moving in the linear motion direction to a treatment portion configured to perform a medical treatment, a conversion portion configured to convert an amount of movement of the driven portion in the linear motion direction to transmit the amount of movement converted to the power transmission portion, and a body storing therein the driven portion and the conversion portion and supporting the treatment portion.

With this configuration, the provision of the conversion portion enables the amount of movement of the driven portion in the linear motion direction to be converted and transmitted to the power transmission portion. For example, it is possible to set a ratio of conversion by the conversion portion depending on the size of, for example, the treatment portion. Specifically, in the case where the size of the treatment portion is relatively small, a conversion ratio is set to reduce the amount of movement produced by the driving force, and in the case where the size of the treatment portion is relatively large, a conversion ratio is set to increase the amount of movement produced by the driving force.

This configuration also makes it easier, even when the size of, for example, the treatment portion is changed, to maintain the relationship between the amount of movement transmitted from outside to the driven portion and the amount of operation of the treatment portion. This configuration also makes it possible to inhibit fluctuations in the S/N ratio and fluctuations in the resolution due to a change in size of, for example, the treatment portion, thus enabling smooth control of operation of the treatment portion and inhibiting deterioration in accuracy in estimating the external force applied to the treatment portion.

As a result, it is easier to achieve safety and to inhibit complications in robot surgery with the surgical tool according to various embodiments. In addition, this configuration facilitates improvement in quality of life (QOL) of patients and facilitates reduction of burden on doctors during surgery. Furthermore, this configuration facilitates improvement in the learning curve in robot surgery with the surgical tool.

The surgical tool may further comprise a connection portion arranged between the power transmission portion and the conversion portion, and the connection portion may be configured to transmit the driving force transmitted from the conversion portion to the power transmission portion. The provision of the connection portion makes transmission of the driving force from the conversion portion to the power transmission portion easier as compared to a configuration in which the driving force is directly transmitted to the power transmission portion. The provision of the connection portion also makes the setting easier for converting the amount of movement in the linear motion direction into a specified converted amount.

The conversion portion may be formed in an elongated shape comprising a first end rotatably supported with respect to the driven portion and a second end rotatably supported with respect to a supporting portion supporting the conversion portion, and the connection portion may be rotatably supported with respect to the conversion portion between the first end and the second end. This shape of the conversion portion makes the setting easier for reducing the amount of movement in the linear motion direction at a specified conversion ratio. In other words, it is possible to change the conversion ratio of the amount of movement in the linear motion direction by changing the ratio between a distance from the position at which the conversion portion is rotatably supported with respect to the driven portion to the position at which the connection portion is rotatably supported, and a distance from the position at which the conversion portion is rotatably supported with respect to the support portion to the position at which the connection portion is rotatably supported.

The conversion portion may be formed in an elongated shape comprising a first end rotatably supported with respect to the driven portion and a second end rotatably supported with respect to the connection portion, and a portion of the conversion portion between the first end and the second end may be rotatably supported with respect to a support portion supporting the conversion portion. This shape of the conversion portion makes the setting easier for reducing the amount of movement in the linear motion direction or for increasing the amount of movement at a specified conversion ratio. In other words, it is possible to change the conversion ratio of the amount of movement in the linear motion direction by changing the ratio between a distance from the position at which the conversion portion is rotatably supported with respect to the driven portion to the position at which the conversion portion is rotatably supported with respect to the support portion, and a distance from the position at which the conversion portion is rotatably supported with respect to the connection portion to the position at which the conversion portion is rotatably supported with respect to the support portion.

In the conversion portion, a first distance from a position at which the conversion portion is rotatably supported with respect to the driven portion to a position at which the support portion is rotatably supported may be greater than a second distance from a position at which the conversion portion is rotatably supported with respect to the connection portion to the position at which the support portion is rotatably supported. When the first distance is greater than the second distance in the conversion portion, the amount of movement of the driven portion in the linear motion direction may be converted into a smaller amount and transmitted to the power transmission portion. Moreover, the magnitude of the driving force may be converted into a larger magnitude to be transmitted to the power transmission portion.

In the conversion portion, a first distance from a position at which the conversion portion is rotatably supported with respect to the driven portion to a position at which the support portion is rotatably supported may be smaller than a second distance from a position at which the conversion portion is rotatably supported with respect to the connection portion to the position at which the support portion is rotatably supported. When the first distance is smaller than the second distance in the conversion portion, the amount of movement of the driven portion in the linear motion direction may be converted into a larger amount and transmitted to the power transmission portion. Moreover, the magnitude of a driving force may be converted into a smaller magnitude to be transmitted to the power transmission portion.

According to the surgical tool of various embodiments, the provision of the conversion portion enables the amount of movement of the driven portion in the linear motion direction to be converted and transmitted to the power transmission portion, thus making it possible to inhibit deterioration in operability and possible to increase accuracy in estimating an external force due to a change in size of the surgical tool.

Various embodiments of the surgical tool will now be described with reference to the drawings.

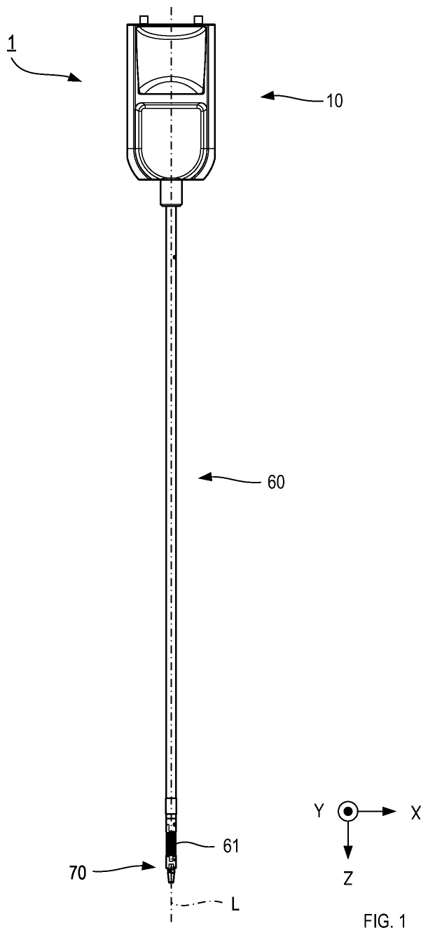

FIGS. 1-7 illustrate examples of a surgical tool according to various embodiments. A surgical tool 1 may be used for master-slave type surgical robots.

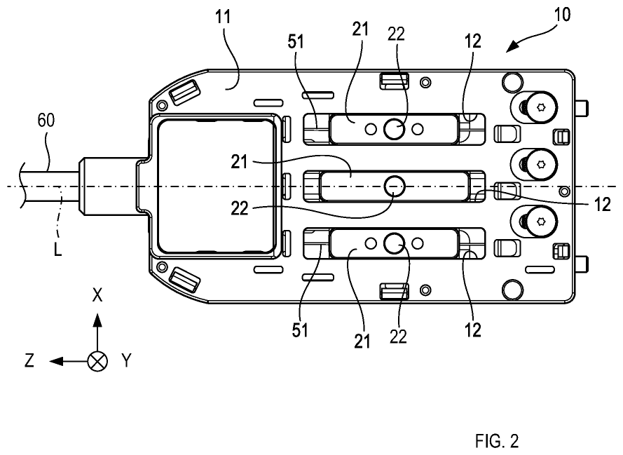

As shown in FIG. 1 and FIG. 2, the surgical tool 1 comprises a body 10, a shaft 60, a joint portion 61, and a forceps 70. The body 10 has a storage space therein. The shaft 60 extends in a rod shape from the body 10, and the forceps 70 is arranged at an end of the shaft 60 opposite from the body 10. The forceps 70 corresponds to one example configuration of a treatment portion. In other embodiments, the treatment portion may be any kind of treatment tool, such as, for example, a scissors, monopolar hooks, spatulas, etc.

For the purpose of simplifying the description, a direction along an axis line L of the shaft 60 will be described as a Z-axis, and a direction from the body 10 toward the forceps 70 will be described as a positive direction of the Z-axis. A direction orthogonal to the Z-axis and parallel to a paper surface of FIG. 1 will be described as an X-axis, and a rightward direction with respect to the positive direction of the Z-axis will be described as the positive direction of the X-axis. A direction orthogonal to the X-axis and the Z-axis will be described as a Y-axis, and a direction from a paper surface of FIG. 1 toward a viewer will be described as a positive direction of the Y-axis.

As shown in FIG. 1, the shaft 60 may be a cylindrically formed member arranged to extend from the body 10 along the Z-axis direction. The forceps 70 are arranged in the end portion of the shaft 60 in the positive direction of the Z-axis. The shaft 60 comprises the joint portion 61 near the forceps 70.

The joint portion 61 is configured to allow changes in orientation of the forceps 70, and is rotatable about a rotational axis in the X-axis direction and a rotational axis in the Y-axis direction. The joint portion 61 is configured to be rotated by, for example, a driving force transmitted by a surgical robot. The configuration of the joint portion 61 is not particularly limited.

The forceps 70 are arranged in the end portion of the shaft 60 in the positive direction of the Z-axis. The forceps 70 are configured to be opened and closed by a driving force transmitted by the surgical robot. The configuration for the opening/closing action of the forceps 70 is not particularly limited.

The body 10 is a portion of the surgical tool 1 that is attached/detached to/from a surgical robot, and is also a portion supporting the shaft 60. In some embodiments, the surgical robot may be a master-slave type surgical robot. As shown in FIG. 2, an attachment/detachment surface 11 of the body 10, which is attached/detached to/from the surgical robot, comprises driven side slits 12 that are elongated holes extending along a Z-axis direction and are driven by an external force from the surgical robot. In the embodiment illustrated in FIG. 2, the surface located on the side in the negative direction of the Y-axis is the attachment/detachment surface 11.

In the driven side slits 12, a plurality of driven portions 21, which will be described later, are arranged to be linearly movable with respect to the body 10 along the Z-axis direction. In the embodiment illustrated in FIG. 2, three driven side slits 12 are aligned at intervals along an X-axis direction. According to various embodiments, the lengths of the three driven side slits 12 along the Z-axis direction may be the same, or the lengths of two of the driven side slits 12 may be the same and the length of one of the driven side slits 12 may be different, or the lengths of all the driven side slits 12 may be different. In various embodiments, the number of the driven side slits 12 that the body 10 comprises may be more than three, or may be less than three.

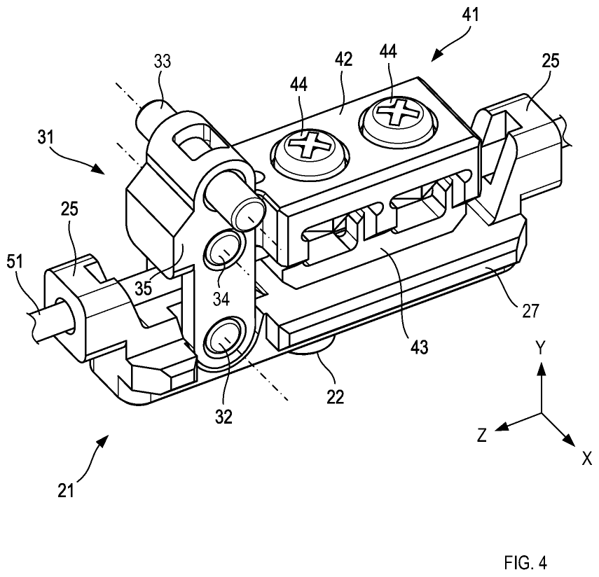

As shown in FIGS. 2 to 4, the body 10 comprises, inside thereof, the driven portions 21, conversion portions 31, connection portions 41, and power transmission portions 51. It is noted that, for example, FIG. 4 only illustrates the configuration of one of each of the driven portions 21, the conversion portions 31, the connection portions 41, and the power transmission portions 51 for ease of description, but each of the driven portions 21 is provided with a corresponding conversion portion 31, a corresponding connection portion 41, and a corresponding transmission portion 51. The driven portions 21 are used for transmitting a driving force for moving, for example, the forceps 70, the conversion portions 31, the connection portions 41, and the power transmission portions 51.

The driven portions 21 receive from the surgical robot a driving force for moving the forceps 70 and the like. As shown in FIG. 2, the driven portions 21 are arranged to be linearly movable within the driven side slits 12 along the Z-axis direction in accordance with the driving force transmitted from the surgical robot.

Each of the driven portions 21 comprises a protrusion 22, driven side openings 23, guide portions 25, through holes 26, and slide guides 27.

The protrusion 22 is a columnar portion that protrudes from the driven portion 21 in the negative direction of the Y-axis, and, when the driven portion 21 is arranged within the driven side groove 12 (as shown in FIG. 2), the protrusion 22 protrudes in the negative direction of the Y-axis further than the attachment/detachment surface 11. The protrusion 22 is configured to engage with a recess formed in a component of the surgical robot (not shown) that transmits a driving force. This engagement allows transmission of the driving force for producing linear motion along the Z-axis direction.

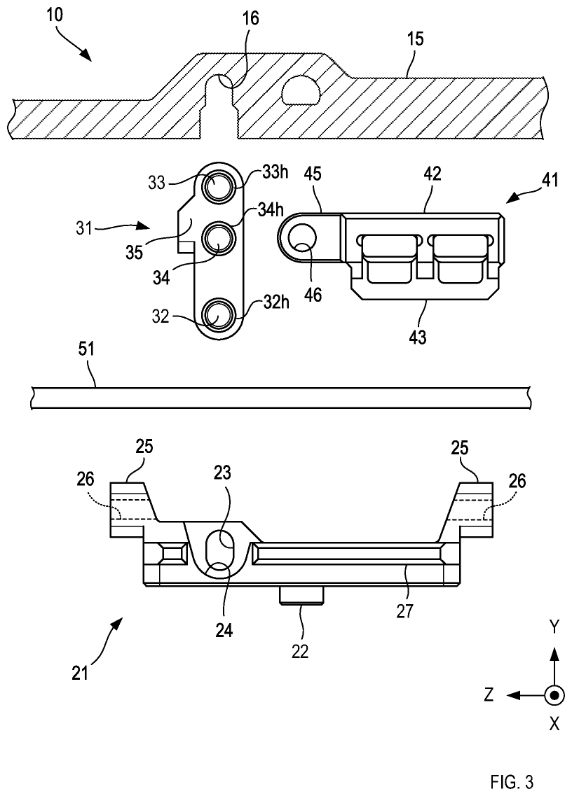

As shown in FIG. 3, the driven side openings 23 are connected to the conversion portion 31 such that the conversion portion 31 is rotatable. Each of the driven side opening 23 is a through-hole which extends along the X-axis direction and accommodates a driven shaft 32 of the conversion portion 31 inserted therein. A cross-section of the driven side opening 23, that is a cross-section cut along a plane orthogonal to the X-axis, has an elliptical shape with a long axis of the ellipse extending along the Y-axis direction. A cutout portion 24 may be formed around the driven side opening 23 of the driven portion 21 to allow rotational movement of the conversion portion 31 along the cutout portion 24.

As shown in FIG. 3 and FIG. 4, each of the guide portions 25 has the through hole 26 in which the power transmission portion 51 is inserted. A gap is formed between the through hole 26 and the power transmission portion 51, allowing relative movement between the driven portion 21 and the power transmission portion 51 along the Z-axis direction. In other words, the through holes have a diameter greater than a diameter of the power transmission portion 51. In the embodiment illustrated in FIGS. 3-4, the guide portions 25 are formed at the end of the driven portion 21 in the positive direction of the Z-axis and the end in the negative direction of the Z-axis, protruding in the positive direction of the Y-axis.

The slide guides 27 protrude from the driven portion 21 in the positive direction of the X-axis and the negative direction of the X-axis, and each slide guide 27 has a ridge shape extending along the Z-axis direction. The slide guides 27 engage with grooves or step shapes formed inside the driven side slit 12 along the Z-axis direction so as to guide movement of the driven portion 21 along the driven side slit 12.

The conversion portion 31 forms a linkage mechanism or a lever mechanism that transmits a driving force that is transmitted by the surgical robot to the driven portion 21, to the connection portion 41. The conversion portion 31 of various embodiments reduces an amount of movement of the driven portion 21 along a linear motion direction, i.e., along the Z-axis direction, and transmits the reduced amount of movement to the connection portion 41. The conversion portion 31 also increases the magnitude of a driving force in the driven portion 21 and transmits the driving force with the increased magnitude to the connection portion 41.

The conversion portion 31 comprises a lever 35 that is an elongated member extending at least along the Y-axis direction. In one end portion of the conversion portion 31 on a negative direction side of the Y-axis, that is in the end portion of the lever 35 adjacent to the driven portion 21, a driven shaft hole 32h is formed and a driven shaft 32 is arranged in the driven shaft hole 32h to extend along the X-axis direction. In the end portion of the conversion portion 31 on a positive direction side of the Y-axis, that is an end portion of the lever 35 opposite to the end portion having the driven shaft hole 32h and the driven shaft 32, a support hole 33h is formed and a support shaft 33 supporting the conversion portion 31 is arranged to extend through the support hole 33h along the X-axis direction. Between the driven shaft 32 and the support shaft 33 of the conversion portion 31, a connection shaft hole 34h is formed and a connection shaft 34 is arranged to extend through the connection shaft hole 34h along the X-axis direction. In some embodiments, the driven shaft 32, the support shaft 33 and the connection shaft 34 may each be columnar (e.g., a solid shaft) or cylindrical (e.g., a hollow shaft).

The end portion of the conversion portion 31 adjacent to the driven portion 21 has a bifurcated shape (most easily seen in FIG. 4) in which the bifurcated ends thereof are spaced apart along the X-axis direction and extend in the negative direction of the Y-axis. Between the bifurcated ends, a portion of the driven portion 21 in which the driven side opening 23 and the cutout portion 24 are formed is inserted. In an area of the conversion portion 31 in which the connection shaft 34 is arranged, a recess that is open in the negative direction of the Z-axis is formed to receive part of the connection portion 41 to be described later.

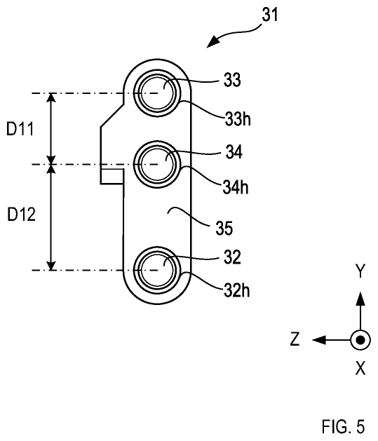

As shown in FIG. 5, in the conversion portion 31, a distance from the center of the support shaft hole 33h (and thus the support shaft 33) to the center of the connection shaft hole 34h (and thus the connection shaft 34) may be a first distance D11. Moreover, in the conversion portion 31, a distance from the center of the driven shaft hole 32h (and thus the driven shaft 32) to the center of the connection shaft hole 34h (and thus the connection shaft 34) may be a second distance D12.

As shown in FIG. 4, the support shaft 33 is arranged to extend in the positive direction of the X-axis and the negative direction of the X-axis beyond the conversion portion 31. As shown in FIG. 3, the support shaft 33 is arranged in a support groove 16 of a support portion 15 of the body 10. The support groove 16 is open in the negative direction of the Y-axis and extends along the X-axis direction.

As shown in FIG. 3 and FIG. 4, the connection portion 41 comprises a first connection portion 42, a second connection portion 43, securing portions 44, a protruding portion 45 and a connection hole 46. The connection portion 41 transmits a driving force transmitted from the conversion portion 31 to the power transmission portion 51.

The first connection portion 42 and the second connection portion 43 hold and fasten the power transmission portion 51 therebetween. The securing portions 44 join the first connection portion 42 and the second connection portion 43. In some embodiments, the securing portions 44 may be screws. However, embodiments are not limited thereto, and in other embodiments, the securing portions 44 may have other structures used for fastening.

The first connection portion 42 comprises the protruding portion 45 which is configured to be inserted into the recess described above that is formed around the portion of the conversion portion 31 in which the connection shaft 34 is arranged. The protruding portion 45 comprises the connection hole 46 that is a through hole extending along the X-axis direction. The connection shaft 34 of the conversion portion 31 is rotatably inserted in the connection hole 46 and the connection shaft hole 34h.

The power transmission portion 51 may be a wire or a rod, and transmits a driving force transmitted from the connection portion 41 to the forceps 70. The power transmission portion 51 is arranged to extend from the inside of the body 10 to the forceps 70 through the shaft 60 along the axis line L.

In the embodiment illustrated in FIGS. 2-4, the power transmission portion 51 is a wire which is a cord-shaped element. According to various embodiments, the power transmission portion 51 may be entirely formed of a wire, or may be, for example, a rod, or a combination of a rod in one part and a wire in another part. Embodiments are not particularly limited. In some embodiments, the rod may be columnar or cylindrical.

The joint portion 61 is configured to be rotated by, for example, a driving force transmitted by the power transmission portion 51. Moreover, the forceps 70 are configured to be opened and closed by a driving force transmitted by the power transmission portion 51.

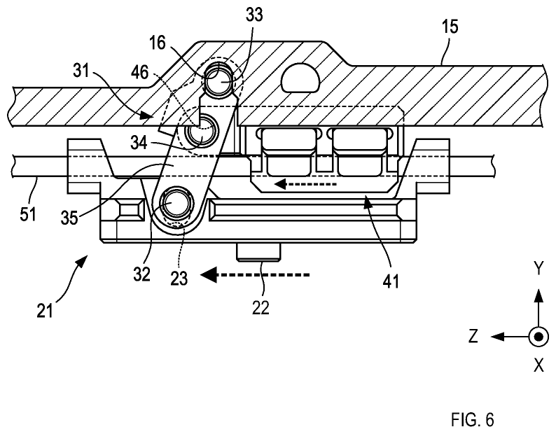

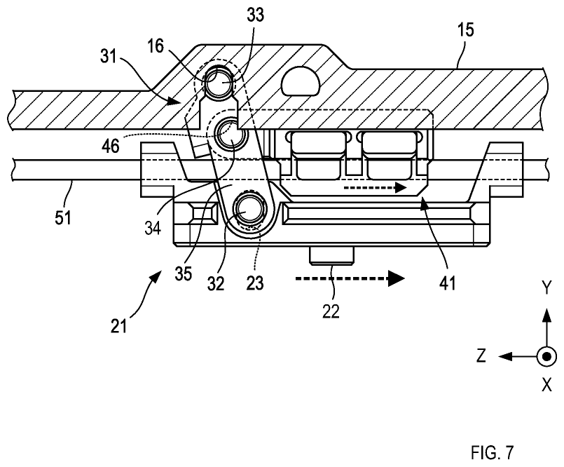

Next, a description will be given of an operation of the surgical tool 1 comprising the above-described configuration with reference to FIG. 6 and FIG. 7. First, the operation when the driven portion 21 is moved in the positive direction of the Z-axis will be described with reference to FIG. 6, and then the operation when the driven portion 21 is moved in the negative direction of the Z-axis will be described with reference to FIG. 7.

As shown in FIG. 6, when the driven portion 21 is linearly moved in the positive direction of the Z-axis (see bottom larger dashed arrow) by a driving force transmitted from the surgical robot, the motion of the driven portion 21 is transmitted to the conversion portion 31. Specifically, the conversion portion 31 is rotated about the support shaft 33 such that an end of the lever 35 adjacent to the driven portion 21 moves in the positive direction of the Z-axis. An amount of movement of the driven shaft 32 of the conversion portion 31 to move in the positive direction of the Y-axis caused by the rotation is absorbed by the driven side opening 23 of the driven portion 21.

When the conversion portion 31 is rotated, the motion of the conversion portion 31 (i.e., the lever 35) is transmitted to the connection portion 41 and the power transmission portion 51. Specifically, the rotation of the conversion portion 31 is converted into and transmitted to movement of the connection portion 41 and the power transmission portion 51 in the positive direction of the Z-axis through the connection shaft 34 that extends through the connection shaft hole 34h of the conversion portion 31 and the connection hole 46 of the protruding portion 45 of the connection portion 41.

The amount of movement of the connection portion 41 and the power transmission portion 51 in the positive direction of the Z-axis (see top smaller dashed arrow) is reduced as compared to the amount of movement of the driven portion 21. For example, the amount of movement of the connection portion 41 and the power transmission portion 51 is reduced to a value obtained by multiplying the amount of movement of the driven portion 21 in the positive direction of Z-axis by the first distance D11 and dividing the resulting value by a combined value of the first distance D11 and the second distance D12 (i.e., by D11+D12). Moreover, the magnitude of the driving force acting on the connection portion 41 and the power transmission portion 51 in the positive direction of the Z-axis is increased as compared to the magnitude of the driving force acting on the driven portion 21. For example, the magnitude of the driving force acting on the connection portion 41 and the power transmission portion 51 is increased to a value obtained by multiplying the magnitude of the driving force acting on the driven portion 21 in the positive direction of Z-axis by the combined value of the first distance D11 and the second distance D12 (i.e., by D11+D12) and dividing the resulting value by the first distance D11.

As shown in FIG. 7, when the driven portion 21 is linearly moved in the negative direction of the Z-axis by a driving force transmitted from the surgical robot, the conversion portion 31 is rotated about the support shaft 33 such that the end of the lever 35 adjacent to the driven portion 21 moves in the negative direction of the Z-axis. The rotation of the conversion portion 31 (i.e., the lever 35) is converted into movement of the connection portion 41 and the power transmission portion 51 in the negative direction of the Z-axis through the connection shaft hole 34h of the conversion portion 31 and the connection hole 46 of the protruding portion 45 of the connection portion 41.

The amount of movement of the connection portion 41 and the power transmission portion 51 in the negative direction of the Z-axis and the magnitude the driving force acting in the negative direction change in a similar manner as in the case shown in FIG. 6; accordingly the detailed description thereof is omitted for conciseness.

According to the surgical tool 1 configured as illustrated in FIGS. 1-7, the provision of the conversion portion 31 enables the amount of movement of the driven portion 21 in the linear motion direction to be converted and transmitted to the power transmission portion 51. For example, it is possible to set the ratio of conversion by the conversion portion 31 depending on the size of, for example, the forceps 70. Specifically, the conversion ratio is set such that the amount of movement to be transmitted from the driven portion 21 to the power transmission portion 51 is reduced based on the extent of a decrease in size of the forceps 70.

This configuration makes it easier, even when the size of, for example, the forceps 70 is changed, to maintain the relationship between the amount of movement transmitted from the surgical robot to the driven portion 21 and the amount of operation of the forceps 70. In the case where the surgical robot comprises a sensor to detect an external force applied to the forceps 70 and/or a sensor, such as an encoder, to detect a driven amount of the forceps 70, it is possible to inhibit fluctuations in the S/N ratio and fluctuations in the resolution due to a change in size of, for example, the forceps 70. This configuration thus enables smooth control of the operation of the forceps 70 and inhibits deterioration in accuracy in estimating an external force applied to the forceps 70.

As a result, it is easier to achieve safety and to inhibit complications in robot surgery with the surgical tool 1 according to various embodiments. In addition, this configuration facilitates improvement in Quality of Life (QOL) of patients and facilitates a reduction of burden on doctors during surgery. Furthermore, this configuration facilitates improvement in the learning curve in robot surgery with the surgical tool 1.

The provision of the connection portion 41 makes transmission of a driving force to the power transmission portion 51 easier as compared to a configuration in which a driving force is directly transmitted from the conversion portion 31 to the power transmission portion 51. This configuration also makes the setting easier for converting the amount of movement in the linear motion direction into a specified converted amount.

The shape of the conversion portion 31 in the embodiment illustrated in FIGS. 1-7 makes the setting easier for reducing the amount of movement in the linear motion direction at a specified conversion ratio. In other words, it is possible to change the conversion ratio of the amount of movement in the linear motion direction by changing the ratio between the first distance D11, which is from the position at which the conversion portion 31 is rotatably supported with respect to the support portion 15 to the position at which the connection portion 41 is rotatably supported, and the second distance D12, which is from the position at which the conversion portion 31 is rotatably supported with respect to the driven portion 21 to the position at which the connection portion 41 is rotatably supported.

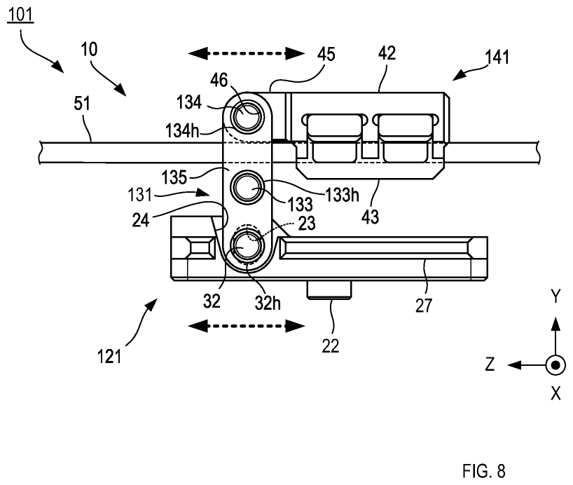

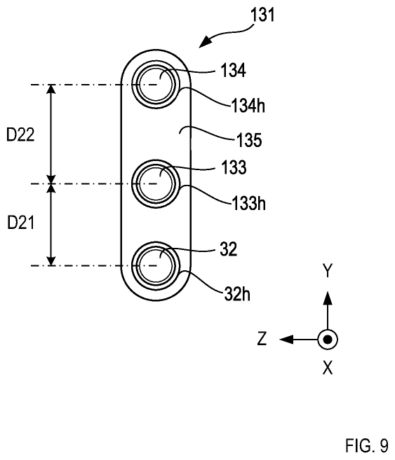

FIGS. 8-9 illustrate a surgical tool according to various embodiments. In the description of FIGS. 8-9, like reference numbers refer to like elements/components and a repeated description thereof is omitted for conciseness. The description will be given with regard to the configuration of the conversion portion and the surrounding components thereof with reference to FIG. 8 and FIG. 9, and the description of other components will not be repeated for conciseness.

As shown in FIG. 8, the body 10 of a surgical tool 101 comprises a driven portion 121, a conversion portion 131, a connection portion 141, and the power transmission portion 51. The connection portion 141 is arranged at the end of the conversion portion 131 in the positive direction of Y-axis, rather than being arranged between the support shaft and the driven shaft.

The driven portion 121 comprises the protrusion 22, the driven side openings 23, and the slide guides 27. The driven portion 121 also comprises the cutout portions 24. In the embodiment illustrated in FIGS. 8-9, the driven portion 121 omits the guide portions 25 and the through holes 26. However, embodiments are not limited to this configuration and, in some embodiments, the driven portion 121 may comprise the guide portions 25 and the through holes 26.

The conversion portion 131 forms a linkage mechanism or a lever mechanism that transmits a driving force that is transmitted by the surgical robot to the driven portion 121, to the connection portion 141. The conversion portion 131 according to various embodiments reduces or increases the amount of movement of the driven portion 121 along the linear motion direction, i.e., along the Z-axis direction, and transmits the reduced or increased amount of movement to the connection portion 141. The conversion portion 131 also increases or reduces the magnitude of a driving force in the driven portion 121 and transmits the driving force with the increased or reduced magnitude to the connection portion 141.

The conversion portion 131 comprises a lever 135 that is an elongated member extending at least along the Y-axis direction. In the end portion of the conversion portion 131 on the negative direction side of the Y-axis, that is in the end portion of the lever 135 adjacent to the driven portion 121, the driven shaft hole 32h is formed and the driven shaft 32 is arranged in the driven shaft hole 32h to extend along the X-axis direction. In the end portion of the conversion portion 131 on the positive direction side of the Y-axis, that is, an end portion of the lever 35 opposite to the end portion having the driven shaft hole 32h and the driven shaft 32, a connection shaft hole 134h is formed and a connection shaft 134 is arranged in the connection shaft hole 134h to extend along the X-axis direction.

Between the driven shaft 32 and the connection shaft 134 of the conversion portion 131, a support shaft hole 133h is formed and a support shaft 133 supporting the conversion portion 131 is arranged in the support shaft hole 133h to extend along the X-axis direction. The support shaft 133 is rotatably held with respect to the support portion 15 of the body 10. In some embodiments, the driven shaft 32, the support shaft 133 and the connection shaft 134 may each be columnar (e.g., a solid shaft) or cylindrical (e.g., a hollow shaft).

As shown in FIG. 9, in the conversion portion 131, a distance from the center of the driven shaft hole 32h (and thus the driven shaft 32) to the center of the support shaft hole 133h (and thus the support shaft 133) is a first distance D21. Moreover, in the conversion portion 131, a distance from the center of the support shaft hole 133h (and thus the support shaft 133) to the center of the connection shaft hole 134h (and thus the connection shaft 134) is a second distance D22.

As shown in FIG. 8, the connection portion 141 transmits a driving force transmitted from the conversion portion 131 to the power transmission portion 51. In comparison with the connection portion 41 in the embodiment illustrated in FIGS. 1-7, the connection portion 141 is connected to the connection shaft 134 of the conversion portion 131 such that the connection shaft 134 is rotatable. That is, the protruding portion 45 and the connection hole 46 and positioned such that an axis of the connection hole 46 aligns with the connection shaft hole 134h and the connection shaft 134 extends through the connection hole 46 and the connection shaft hole 134h.

Next, a description will be given of an operation of the surgical tool 101 comprising the configuration illustrated in FIG. 8, with reference to FIG. 8.

When the driven portion 121 is linearly moved in the positive direction of the Z-axis by a driving force transmitted from the surgical robot, the motion of the driven portion 121 is transmitted to the conversion portion 131. Specifically, the conversion portion 131 is rotated about the support shaft 133 such that the end portion of the lever 135 adjacent to the driven portion 121 moves in the positive direction of the Z-axis.

When the conversion portion 131 is rotated, the motion of the conversion portion 131 is transmitted to the connection portion 141 and the power transmission portion 51. Specifically, the rotation of the conversion portion 131 is converted into movement of the connection portion 141 and the power transmission portion 51 in the negative direction of the Z-axis.

An absolute value of an amount of movement of the connection portion 141 and the power transmission portion 51 in the negative direction of the Z-axis is a value obtained by multiplying an absolute value of an amount of movement of the driven portion 121 in the positive direction of the Z-axis by the second distance D22 illustrated in FIG. 9 and dividing the resulting value by the first distance D21 illustrated in FIG. 9. Moreover, a magnitude of the driving force acting on the connection portion 41 and the power transmission portion 51 in the positive direction of the Z-axis is a value obtained by multiplying the magnitude of the driving force acting on the driven portion 21 in the positive direction of the Z-axis by the first distance D21 and dividing the resulting value by the second distance D22.

When the first distance D21 is greater than the second distance D22, the absolute value of the amount of movement of the connection portion 141 and the power transmission portion 51 in the negative direction of the Z-axis becomes smaller than the absolute value of the amount of movement of the driven portion 121 in the positive direction of the Z-axis. The magnitude of the driving force acting on the connection portion 41 and the power transmission portion 51 in the positive direction of the Z-axis becomes larger than the magnitude of the driving force acting on the driven portion 21 in the positive direction of the Z-axis.

When the first distance D21 is smaller than the second distance D22, the absolute value of the amount of movement of the connection portion 141 and the power transmission portion 51 in the negative direction of the Z-axis becomes larger than the absolute value of the amount of movement of the driven portion 121 in the positive direction of the Z-axis. The magnitude of the driving force acting on the connection portion 41 and the power transmission portion 51 in the positive direction of the Z-axis becomes smaller than the magnitude of the driving force acting on the driven portion 21 in the positive direction of the Z-axis. When the first distance D21 is equal to the second distance D22, the absolute value of the amount of movement of the connection portion 141 and the power transmission portion 51 in the negative direction of the Z-axis is the same as the absolute value of the amount of movement of the driven portion 121 in the positive direction of the Z-axis. The magnitude of the driving force acting on the connection portion 141 and the power transmission portion 51 in the positive direction of the Z-axis is the same as the magnitude of the driving force acting on the driven portion 121 in the positive direction of the Z-axis.

In the configuration illustrated in FIGS. 8-9, the movement of the conversion portion 131, the connection portion 141, and the power transmission portion 51 when the driven portion 121 is linearly moved in the negative direction of the Z-axis by a driving force transmitted from the surgical robot is in a direction opposite to the direction of the movement described above (i.e., is in the positive direction of the Z-axis). Accordingly, a detailed description thereof is omitted for conciseness. Moreover, the amount of movement of the connection portion 141 and the power transmission portion 51 along the Z-axis direction and the magnitude the driving force change in a similar manner, and therefore a detailed description thereof is also omitted for conciseness.

According to the configuration illustrated in FIGS. 8-9, the configuration of conversion portion 131 makes the setting easier for reducing the amount of movement in the linear motion direction or for increasing the amount of movement at a specified conversion ratio. In other words, it is possible to change the conversion ratio of the amount of movement in the linear motion direction by changing the ratio between the first distance D21, which is from the position at which the conversion portion 131 is rotatably supported with respect to the driven portion 121 to the position at which the conversion portion 131 is rotatably supported with respect to the support portion 15, and the second distance D22, which is from the position at which the conversion portion 131 is rotatably supported with respect to the connection portion 141 to the position at which the conversion portion 131 is rotatably supported with respect to the support portion 15.

Setting the first distance D21 greater than the second distance D22 in the conversion portion 131 enables the amount of movement of the driven portion 121 in the linear motion direction to be converted into a smaller amount and transmitted to the power transmission portion 51. Moreover, the magnitude of a driving force is converted into a larger magnitude to be transmitted to the power transmission portion 51.

Setting the first distance D21 smaller than the second distance D22 in the conversion portion 131 enables the amount of movement of the driven portion 121 in the linear motion direction to be converted into a larger amount and transmitted to the power transmission portion 51. Moreover, the magnitude of a driving force is converted into a smaller magnitude to be transmitted to the power transmission portion 51.

It is to be noted that the technical scope of the present disclosure should not be limited to the above-described embodiments, and various modifications thereto may be made without departing from the intent of the present disclosure. For example, in the embodiments described above, the treatment portion arranged at the distal end of the shaft 60 is the forceps 70. The treatment portion, however, is not limited to the forceps 70 and may be other instruments used for, for example, endoscope surgery. For example, the treatment portion may be a scissors, monopolar hooks, spatulas, etc.

In addition, the specific shapes of the conversion portions 31 and 131 are not limited to those described in the above-described embodiments. Any shapes that would achieve the same effect may be used, and thus the shapes are not limited to particular shapes.

It should be understood that the present disclosure is not limited to the above embodiments, but various other changes and modifications may be made therein without departing from the spirit and scope as set forth in appended claims.

What is claimed is:

1. A surgical tool comprising:
a driven portion configured to move in a linear motion direction, based on a driving force;
a power transmission portion configured to transmit the driving force for moving in the linear motion direction to a treatment portion configured to perform a medical treatment;
a conversion portion configured to convert a first amount of movement of the driven portion in the linear motion direction to a second amount of movement different from the first amount of movement and to transmit the second amount of movement to the power transmission portion; and
a body that includes the driven portion and the conversion portion, and that supports the treatment portion.

2. The surgical tool according to claim 1, further comprising a connection portion connected to the power transmission portion and to the conversion portion, the connection portion being configured to transmit the driving force that is transmitted from the conversion portion to the power transmission portion.

3. The surgical tool according to claim 2,
wherein the conversion portion has an elongated shape and comprises a first end rotatably connected to the driven portion and a second end rotatably connected to a supporting portion of the body, and
wherein the connection portion is rotatably connected to the conversion portion between the first end and the second end of the conversion portion.

4. The surgical tool according to claim 2,
wherein the conversion portion has an elongated shape and comprises a first end rotatably connected to the driven portion and a second end rotatably connected to the connection portion, and
wherein a portion of the conversion portion between the first end and the second end is rotatably connected to a supporting portion of the body.

5. The surgical tool according to claim 4,
wherein a first distance from a first position at which the conversion portion is rotatably connected to the driven portion to a second position at which the supporting portion is rotatably connected to the conversion portion is greater than a second distance from the second position to a third position at which the conversion portion is rotatably connected to the connection portion.

6. The surgical tool according to claim 4,
wherein a first distance from a first position at which the conversion portion is rotatably connected to the driven portion to a second position at which the supporting portion is rotatably connected to the conversion portion is less than a second distance from the second position to a third position at which the conversion portion is rotatably connected to the connection portion.

7. The surgical tool according to claim 3,
wherein a first distance from a first position at which the conversion portion is rotatably connected to the driven portion to a second position at which the conversion portion is rotatably connected to the connection portion is greater than a second distance from the second position to a third position at which the supporting portion is rotatably connected to the conversion portion.

8. The surgical tool according to claim 1, wherein the second amount of movement is less than the first amount of movement.

9. The surgical tool according to claim 1, wherein the second amount of movement is greater than the first amount of movement.

10. The surgical tool according to claim 1, wherein the conversion portion is configured to convert a first magnitude of the driving force received by the driven portion into a second magnitude of the driving force that is greater than the first magnitude of the driving force and to transmit the second magnitude of the driving force to the power transmission portion.

11. A surgical tool comprising:
a shaft;
a treatment portion provided at a distal end of the shaft; and
a body provided at a proximal end of the shaft, wherein the body comprises:
a driven portion that moves in direction parallel to the shaft, based on a first driving force from a surgical robot;
a wire or rod that is connected through the shaft to the treatment portion;
a lever that converts the first driving force to a second driving force, the second driving force being different from the first driving force; and
a connection portion that is connected to the lever and to the wire or rod, and that transmits the second driving force to the wire or rod.

12. The surgical tool of claim 11, wherein a first magnitude of the first driving force is less than a second magnitude of the second driving force.

13. The surgical tool of claim 12, wherein the lever converts a first amount of movement of the driven portion to a second amount of movement less than the first amount of movement and transmits the second amount of movement to the connection portion.

14. The surgical tool of claim 11, wherein the lever converts a first amount of movement of the driven portion to a second amount of movement less than the first amount of movement and transmits the second amount of movement to the connection portion.

15. The surgical tool of claim 11, wherein the lever has a first end and a second end opposite to the first end, and
the first end of the lever is rotatably connected to the driven portion,
the second end of the lever is rotatably connected to the body; and
the lever is rotatably connected to the connection portion at a point between the first end and the second end.

16. The surgical tool of claim 15, wherein a first distance from a first position at which the lever is rotatably connected to the driven portion to a second position at which the lever is rotatably connected to the connection portion is greater than a second distance from the second position to a third position at which the lever is rotatably connected to the body.

17. The surgical tool of claim 11, wherein the lever has a first end and a second end opposite to the first end, and
the first end of the lever is rotatably connected to the driven portion,
the second end of the lever is rotatably connected to the connection portion; and
the lever is rotatably connected to the body at a point between the first end and the second end.

18. The surgical tool according to claim 17,
wherein a first distance from a first position at which the lever is rotatably connected to the driven portion to a second position at which the lever is rotatably connected to the body is greater than a second distance from the second position to a third position at which the lever is rotatably connected to the connection portion.

19. The surgical tool according to claim 17,
wherein a first distance from a first position at which the lever is rotatably connected to the driven portion to a second position at which the lever is rotatably connected to the body is less than a second distance from the second position to a third position at which the lever is rotatably connected to the connection portion.

20. The surgical tool according to claim 11, further comprising a joint portion provided between the shaft and the treatment portion.
